(19) United States
(12) Patent Application Publication
(10) Pub. No.: US 2008/0014161 A1
(43) Pub. Date: Jan. 17, 2008

(54) COSMETIC COMPOSITIONS COMPRISING POLYMERS COMPRISING COMPLEMENTARY CHEMICAL FUNCTIONAL GROUPS

(75) Inventors: Henri Samain, Bievres (FR); Isabelle Rollat-Corvol, Paris (FR); Gabin Vic, Compiegne (FR); Aude Livoreil, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 11/778,456

(22) Filed: Jul. 16, 2007

Related U.S. Application Data
(62) Division of application No. 10/321,355, filed on Dec. 18, 2002.

(30) Foreign Application Priority Data
Dec. 18, 2001 (FR) 01 16387

(51) Int. Cl. A61K 8/72; A61Q 5/00

(52) U.S. Cl. 424/70.1

(57) ABSTRACT

The invention relates to cosmetic compositions comprising at least two polymers comprising complementary chemical functional groups, capable of forming a coat on a keratin material, such as hair, a cosmetic process comprising the use of these polymers on the keratin material, such as the hair, and also their use for producing a coat on the hair.

This is a division of application Ser. No. 10/321,355, filed Dec. 18, 2002 which claims priority to French Patent Application No. 01 16387, filed Dec. 18, 2001 which are incorporated herein by reference.

Disclosed herein are cosmetic compositions comprising at least two polymers comprising complementary chemical functional groups, capable of forming a coat on a keratin material, such as, hair. Also disclosed herein is a cosmetic process comprising the use of such polymers on keratin material, such as hair, and also their use for coating the hair.

Cosmetic products intended for treating the hair often use polymers. They make it possible to obtain, for example, hairstyle holding effects, softness effects, or sheen effects. However, polymers deposited on the hair may be removed rapidly during shampooing. It thus may be necessary to reapply the product, for example, after each shampoo wash.

There is thus a need to produce cosmetic products that are improved compared with those of the prior art, for example, cosmetic products that are remanent with respect to repeated washing.

The inventors have discovered, surprisingly and unexpectedly, that it is possible to achieve at least one of the objectives listed above by selecting the polymers introduced into the cosmetic products on the basis of the nature of the chemical functional groups they bear, in order to be applied to keratin material, such as hair.

Thus, disclosed herein is a cosmetic composition comprising at least one polymer PA and at least one polymer PB comprising, respectively, chemical functional groups derived from non-photoactivatable groups A and B, which may be identical or different, characterized in that:

(i) the chemical functional groups derived from the groups A and B are complementary chemical functional groups, and

(ii) the polymers PA and PB:

(a) do not react together to form covalent bonds before applying the cosmetic composition to a keratin material, such as hair, and

(b) are capable of reacting to form covalent bonds together after applying the cosmetic composition to a keratin material, such as hair.

Also disclosed herein is a cosmetic process comprising the application of the composition described herein.

Further disclosed herein is a cosmetic process comprising the application, separately or sequentially over time, to a keratin material, such as hair, of at least two compositions, wherein one of them comprises at least one polymer PA, and the other comprises at least one polymer PB, respectively, wherein the at least one polymer PA and the at least one polymer PB are polymers comprising complementary chemical functional groups.

Still further disclosed herein is the use of these processes for producing a coat on hair.

To determine whether polymers PA and PB, comprising chemical functional groups derived from the groups A and B, respectively, comprise complementary chemical functional groups, the test described below is carried out, wherein operations (1) to (4) are performed at room temperature:

(1) 0.25 gram of a solution of the polymer PA in a solvent chosen from water, ethanol, esters and ketones, for example, water, wherein the weight content of the polymer PA ranges from 10% to 50% relative to the total weight of the solution, is applied to a glass slide;

(2) the solvent for the polymer PA is allowed to evaporate off until a dry deposit is obtained;

(3) 0.25 gram of a solution of the polymer PB in a solvent chosen from water, ethanol, esters and ketones, for example, water, wherein the weight content of the polymer PB ranges from 10% to 50% relative to the total weight of the solution, is applied to the slide already covered with the polymer PA;

(4) the solvent for the polymer PB is allowed to evaporate off until a dry deposit is obtained;

(5) the glass slide covered with the polymers PA and PB is placed in a chamber at a temperature of 100° C. for a period of 120 minutes; (6) in the case where the solvents used in (1) and (3) are different, (1) through (5) are repeated a second time;

(7) the solid obtained is completely immersed in 10 grams of the solvent used in (1); (8) in the case where the solvents used in (1) and (3) are different, the solid obtained from (6) is completely immersed in 10 grams of the solvent used in (3);

(9) the polymers PA and PB are termed “polymers comprising complementary chemical functional groups” if at least 50% by weight of the solid does not dissolve after 3 days, at room temperature and without stirring, in the solvents of (7) and (8).

In (1) and (3), when the solutions are aqueous, the pHs of the solutions are, for example, adjusted, to the pH at which the compositions of the invention are used.

Without wishing to be bound by the theory, the inventors believe that the insoluble solid deposit may result from the formation of covalent bonds between the polymers PA and PB (e.g., substitution reactions, addition reactions to carbon-carbon or carbon-hetero atom double or triple bonds, or ring-opening reactions). And characterization techniques known to those skilled in the art, such as infrared or ESCA (XPS) spectroscopy may be used to evaluate, depending on the case, if and to what extent the formation of these covalent bonds may have taken place.

The chemical functional groups of the group A are chosen from the following functional groups:

epoxide,

aziridine,

vinyl and activated vinyl, such as, acrylonitrile, acrylic and methacrylic esters, crotonic acid and esters, cinnamic acid and esters, styrene and derivatives, butadiene, vinyl ethers, vinyl ketone, maleic esters, vinyl sulphones and maleimides,

anhydride, acid chloride and carboxylic acid esters,

aldehydes,

acetals, hemiacetals,

aminals, hemiaminals,

ketones, α-hydroxy ketones, α-halo ketones,

lactones, thiolactones,

isocyanate,

thiocyanate,

imines,

imides, such as succinimide and glutimide,

N-hydroxysuccinimide esters,

imidates,

thiosulphate,

oxazine and oxazoline,

oxazinium and oxazolinium,

C1 to C30 alkyl halides, C6 to C30 aryl and aralkyl halides of formula RX, wherein X is chosen from I, Br and Cl,

halides of unsaturated carbon-based rings and heterocycles, for example, chlorotriazine, chloropyrimidine, chloroquinoxaline and chlorobenzotriazole, and

sulphonyl halide, such as RSO₂Cl and RSO₂F, wherein R is chosen from C1 to C30 alkyls.

The chemical functional groups of the group B are chosen from the functional groups of XHₙ, wherein X is chosen from O, N, S, and COO, and n=1 or 2, for example, alcohol, amine, thiol and carboxylic acid.

The cosmetic composition disclosed herein excludes polymers comprising photoactivatable chemical functional groups, i.e. polymers comprising chemical functional groups which, when irradiated with a wavelength ranging from 200 to 800 nm, give rise, in at least one step, to the formation of new covalent bonds.

The product as disclosed herein may thus be in the form of a composition comprising a combination of at least two polymers PA and PB, comprising complementary chemical functional groups, in a cosmetically acceptable medium, wherein the cosmetically acceptable medium is chosen such that the at least two polymers remain inert with respect to each other in the composition, but react, forming covalent bonds, after applying the composition to hair.

The product as disclosed herein may also be in the form of a combination of compositions, for example, a kit comprising at least two compartments, the first compartment comprising a composition comprising at least one polymer PA in a cosmetically acceptable medium, and the second compartment comprising at least one polymer PB in a cosmetically acceptable medium, wherein PA and PB have complementary chemical functional groups, and the cosmetically acceptable mediums may be identical or different.

When the product as disclosed herein is in the form of a combination of compositions, such as a kit, the compositions comprising at least one polymer PA and at least one polymer PB, respectively, are used separately or sequentially over time, wherein at least one application of these compositions may possibly take place.

The chemical functional groups of the group A may react with the chemical functional groups of the group B, either spontaneously or via an activator chosen from temperature, pH, at least one co-reagent, and at least one catalyst chosen from chemical and biochemical catalysts, for instance an enzyme.

For example, the chemical functional groups of the group A can be chosen from anhydride, epoxide, chlorotriazine, aldehyde and thiosulphate functional groups, and the chemical functional groups of the group B can be chosen from hydroxyl, primary and secondary amine, thiol and carboxylic acid functional groups.

In one embodiment, each polymer PA or PB comprises at least two identical chemical functional groups chosen from those of the group A and those of the group B, respectively.

The polymers as disclosed herein may contain chemical functional groups in addition to those of groups A and B.

In one embodiment, the polymers comprising anhydride chemical functional groups do not comprise carboxylic acid functional groups.

As disclosed herein, the term “polymer” means a compound comprising at least five repeating units linked by covalent bonds.

The polymers may be synthesized

by free-radical reactions (for example, polyacrylates, polymethacrylates, polyvinyls, etc.),

by condensation reactions (for example, polyesters, polyethers, polyamides, polyurethanes, polydimethylsiloxanes, polypeptides, etc.), and

by ring-opening reactions (for example, polyesters, etc.).

The polymers may be of an origin chosen from natural, chemically modified and unmodified origins, such as polysaccharides (cellulose, dextran, chitosan, guar and the hydroxyalkyl, carboxymethyl, amino and thiol derivatives thereof, and derivatives thereof comprising functional groups chosen from aldehyde and epoxy).

The polymers may be in any type of topology chosen from linear, branched, starburst and hyperbranched (for example, dendrimers) chains, and block, random, and alternating chains.

The chemical functional groups may be naturally present on the polymer chain, at the end of the chain, grafted along the main chain or the secondary chains, or on the branches of starburst or hyperbranched polymers.

Each polymer comprises at least one chemical functional group of the group A or B.

In one embodiment, each polymer contains at least two identical chemical functional groups (A,A or B,B), in order to bond with at least two other polymers.

The combinations of polymers are, for example, as follows:

dendrimers comprising end groups chosen from OH, NH₂, SH and COOH of at least one generation in combination with at least one polymer chosen from polymers comprising functional groups chosen from anhydride units, such as maleic anhydride, and polymers comprising at least one group chosen from epoxide and aldehyde groups

synthetic polymers comprising hydroxyl functional groups, such as polyvinyl alcohols, in combination with at least one polymer chosen from polymers comprising functional groups chosen from anhydride units, such as maleic anhydride, and polymers comprising epoxide groups,

polyethyleneimines in combination with at least one polymer chosen from polymers comprising functional groups chosen from anhydride units, such as maleic anhydride, and polymers comprising at least one group chosen from epoxide and aldehyde groups,

polyethyleneimine thiols, obtained by reacting polyethyleneimines with γ-butyrolactone, such as those described in the patent FR 2 772 770, in combination with at least one polymer chosen from polymers comprising functional groups chosen from anhydride units, such as maleic anhydride, and polymers comprising at least one group chosen from epoxide and aldehyde groups

polyamino acids comprising functional groups chosen from free OH, NH₂, SH and COOH groups, for example polylysine, in combination with at least one polymer chosen from polymers comprising functional groups chosen from anhydride units, such as maleic anhydride, and polymers comprising at least one group chosen from epoxide and aldehyde groups,

polysaccharides chosen from natural and modified polysaccharides comprising functional groups chosen from OH, NH₂, SH and COOH, in combination with at least one polymer chosen from polymers comprising functional groups chosen from anhydride units, such as maleic anhydride, and polymers comprising at least one group chosen from epoxide and aldehyde groups,

polysaccharides chosen from natural and modified polysaccharides comprising aldehyde functional groups in combination with polymers chosen from synthetic and natural polymers comprising functional groups chosen from OH, NH₂, SH and COOH, such as polyethyleneimine, polylysine, chitosan and its derivatives, such as carboxymethylchitosans, and aminodextrans,

polysaccharides chosen from natural and modified polysaccharides comprising epoxy functional groups in combination with polymers chosen from synthetic and natural polymers comprising functional groups chosen from OH, NH₂, SH and COOH, for example, polyethyleneimine, polylysine, chitosan and its derivatives, such as carboxymethylchitosans, and aminodextrans, and

polymers chosen from natural and synthetic polymers comprising carboxylic acid functional groups in combination with polymers chosen from synthetic and natural polymers comprising functional groups chosen from OH, NH₂ and SH, in the presence of at least one entity chosen from carbodiimides, acids, bases and enzymes, for instance esterase, lipase and protease.

For example and without limitation, the following reactive polymers are mentioned:

Polymers Comprising Functional Groups of the Group A

methyl vinyl ether/maleic anhydride copolymer, sold for example, by ISP under the name Gantrez,

polyglycidyl methacrylate, for example, sold by Polysciences,

glycidyl polydimethylsiloxane, for example, sold by Shinetsu (reference X-2Z-173 FX or DX),

epoxy polyamidoamine, sold, for example, by Hercules under the name Delsette 101 or Kymene 450,

epoxydextran, and

polysaccharides and polyaldehydes obtained by oxidizing polysaccharides with NalO₄ (for example, Bioconjugate Techniques; Hermanson G T, Academic Press, 1996).

Polymers Comprising Functional Groups of the Group B

PAMAM dendrimer, for example, sold by Dendritech, DSM, Sigma-Aldrich (Starburst, PAMAM Dendrimer, G(2, O) from Dendritech),

dendrimer comprising hydroxyl functional groups, for example, sold by Perstorp, DSM (example: HBP TMP core 2 Generation Perstorp),

PEI (polyethyleneimine), for example, sold by BASF under the name Lupasol,

PEI-thiol,

polylysine, for example, sold by Chisso,

HP cellulose, (such as Klucel EF from Aqualon),

aminodextran, sold, for example, by Carbomer,

aminocellulose, for example, those described in WO 01/25283 from BASF,

PVA (polyvinyl acetal), for example, Airvol 540 from Air Products Chemical,

amino PVA, sold, for example, by Carbomer,

chitosan,

CM and HP dextran, sold, for example, by Fluka, and

CM and HP chitosan, sold, for example, by Fluka.

As disclosed herein, all the combinations of at least one polymer of the group A and of at least one polymer of the group B are mentioned as non-limiting examples.

The polymers PA and PB comprising, respectively, chemical functional groups derived from the groups A and B, may react together to form covalent bonds, for example, according to the following processes.

Variants of the Application Processes Disclosed Herein:

a) When the reaction between the polymers PA and PB takes place spontaneously at room temperature, (although such a mixture in a dilute solution is stable), a solution comprising the polymers PA and PB, in a cosmetically acceptable volatile solvent, may be applied directly to a material and, during the evaporation of the solvent, the crosslinking reaction can take place. The polymer deposit can become insoluble and remain on the material.

b) When the reaction between the polymers PA and PB requires an activation, such polymer mixture may be applied to a material and, by using at least one activator chosen from increasing the temperature, adding at least one pH modifier, and adding at least one entity chosen from co-reagents and catalysts, the deposit can be made to crosslink.

c) In one embodiment, when one of the polymers PA and PB has particular affinity for a material, this first polymer is deposited on the material first via a cosmetically acceptable volatile solvent, and the polymer capable of reacting with the first polymer may then be deposited, via a cosmetically acceptable volatile solvent. The chemical reaction may then take place spontaneously in the course of the drying or may be triggered by at least one activator chosen from a supply of heat, a change in pH, and an addition of at least one entity chosen from co-reagents and catalysts. In this case, the product is generally a kit. The crosslinked deposit thus formed can have the advantage of having expected low solubility. In addition, it can have good affinity for the surface of the material, which can ensure better remanence of the deposit as a whole.

For example, this type of deposit in layers may also help preserve the cosmetic or optical properties of the polymer, which constitutes the upper part of the deposit.

According to the above disclosed processes, it is possible to produce multiple superpositions of layers of polymers that crosslink together to achieve the desired type of deposit (in terms of chemical nature, mechanical strength, thickness, appearance, feel, etc.).

The cosmetically acceptable medium, conveying the polymer PA and the polymer PB, is chosen such that the polymers PA and PB comprising complementary chemical functional groups are capable of reacting together to form covalent bonds after applying the cosmetic composition to hair. For example, the cosmetically acceptable medium may further comprise a suitable activator chosen from pH modifiers, co-reagents and catalysts, such as:

a pH modifier chosen from acids and bases, of mineral and organic nature;

a co-reagent chosen from carbodiimide, oxidizing agents and reducing agents; and

a catalyst chosen from enzymes.

The processes disclosed herein comprise the application of the above cosmetic compositions to hair.

For example, a process may comprise at least one additional operation chosen from bringing about a change in pH, bringing about an increase in temperature, adding at least one additive, and rinsing.

In one embodiment, a composition chosen from care, dyeing, permanent-reshaping, hair-makeup, fixing and hairstyle-holding compositions is applied, before applying a product as defined above.

The at least two polymers comprising complementary chemical functional groups are present in a concentration ranging, for example, from 0.05% to 50% by weight, further, for example, from 0.1% to 20% by weight and even further, for example, from 0.25% to 10% by weight, relative to the total weight of the composition as disclosed herein.

As disclosed herein, a composition comprising at least two polymers comprising complementary functional groups may further, for example, comprise at least one conventional cosmetic additive chosen from fixing polymers, thickeners, anionic, nonionic, cationic and amphoteric surfactants, fragrances, preserving agents, sunscreens, proteins, vitamins, provitamins, anionic, nonionic, cationic and amphoteric non-fixing polymers, mineral, plant and synthetic oils, ceramides, pseudoceramides, volatile and non-volatile, linearand cyclic, modified and unmodified silicones, pH regulators, oxidizing agents, reducing agents, inhibitors, catalysts and any other additives conventionally used in cosmetic compositions.

The cosmetically acceptable medium is chosen from water, at least one cosmetically acceptable solvent such as alcohols, esters, ketones and volatile cyclic silicones, and water/solvent mixtures. For example, the at least one cosmetically acceptable solvent may be chosen from C₁-C₄ alcohols.

When the composition as disclosed herein is packaged in an aerosol device, the composition further comprises at least one propellant, which may be chosen from volatile hydrocarbons such as n-butane, propane, isobutane, pentane and halogenated hydrocarbons. Carbon dioxide, nitrous oxide, dimethyl ether (DME), nitrogen and compressed air may also be used as the at least one propellant. For example, dimethyl ether may be used.

The at least one propellant is present in a concentration ranging, for example, from 5% to 90% by weight, further, for example, from 10% to 60% by weight, relative to the total weight of the composition, in the aerosol device.

The products as disclosed herein may be applied to dry or wet hair.

The invention will be illustrated more fully with the aid of the following non-limiting example.

### EXAMPLE

Composition A: Starburst PAMAM dendrimers, generation 2, sold in aqueous solution by Dendritech

Composition B: aqueous 5% solution of Gantrez S-97BF, sold by ISP

Composition C: mixture of the two reagents

composition A: 50 g

composition B: 50 g

Composition D: mixture of the two reagents with a cosmetic active agent (panthenol)

composition A: 45 g

panthenol: 1 g

water: qs 50 g

Cosmetic active agent: panthenol

Protocol in Successive Applications

The process was performed on natural hair, with a lock weighing 2.7 g. 0.3 g of composition A was applied to clean, dry hair, followed by applying 0.25 g of composition B. The deposit was dried using a hair dryer for 45 minutes and the lock was then left for two hours at 100° C.

The deposit thus produced on the material was remanent to 10 shampoo washes.

Just after application and heat treatment, a thick and covering coat was observed, which completely covered the scales of the hair. After 10 shampoo washes, the coat appeared to have thinned, especially at the ends, but still clearly covered the hair.

Protocol in Single Applications

The process was performed on natural hair, with a lock weighing 2.7 g.

0.5 g of composition C was applied to clean, dry hair. The deposit was dried with a hair drier for 45 minutes and the lock was then left for two hours at 100° C.

The deposit thus produced on the material was remanent to 10 shampoo washes.

Inclusion of a Cosmetic Active Agent into the Crosslinked Deposit

The process was performed on natural hair, with a lock weighing 2.7 g.

0.3 g of composition D was applied to clean, dry hair, followed by applying 0.25 g of composition B. The deposit was dried with a hair drier for 45 minutes and the lock was then left for two hours at 100° C.

The deposit thus produced on the material was remanent to 10 shampoo washes and contained panthenol.

1. A cosmetic process comprising applying to keratin material, separately or sequentially over time, at least two compositions, wherein one of the at least two compositions comprises at least one polymer PA, and the other of the at least two compositions comprises at least one polymer PB, respectively, wherein the at least one polymer PA and the at least one polymer PB are polymers comprising complementary chemical functional groups
wherein the chemical functional groups of the group A are chosen from the following functional groups:
epoxide,
aziridine,
vinyl and activated vinyl,
anhydride, acid chloride and carboxylic acid esters,
aldehydes,
acetals, hemiacetals,
aminals, hemiaminals,
ketones, α-hydroxy ketones, α-halo ketones,
lactones, thiolactones,
isocyanate,
thiocyanate,
imines,
imides,
N-hydroxysuccinimide esters,
imidates,
thiosuiphate,
oxazine and oxazoline,
oxazinium and oxazolinium,
C1 to C30 alkyl halides, C6 to C30 aryl and aralkyl halides of formula RX, wherein X is chosen from I, Br and Cl,
halides of unsaturated carbon-based rings and heterocycles,
sulphonyl halide chosen from RSO₂Cl and RSO₂F, and wherein R is chosen from C1 to C30 alkyl groups; and
wherein the chemical functional groups of the group B are chosen from functional groups of XHₙ, wherein X is chosen from O, N, S, and COO, and n=1 or 2.

2. The process according to claim 1, wherein the keratin material is hair.

3. The process according to claim 1, wherein, in defining the chemical functional groups of the group A, the vinyl and activated vinyl are chosen from acrylonitrile, acrylic and methacrylic esters, crotonic acid and esters, cinnamic acid and esters, styrene and derivatives, butadiene, vinyl ethers, vinyl ketone, maleic esters, vinyl sulphones and maleimides.

4. The process according to claim 1, wherein, in defining the chemical functional groups of the group A, the imides are chosen from succinimide and glutimide.

5. The process according to claim 1, wherein, in defining the chemical functional groups of the group A, the halides of unsaturated carbon-based rings and heterocycles are chosen from chlorotriazine, chloropyrimidine, chloroquinoxaline and chlorobenzotriazole.

6. The process according to claim 1, wherein, in defining the chemical functional groups of the group B, the functional groups of XHₙ are chosen from alcohol, amine, thiol and carboxylic acid.

7. The process according to claim 1, wherein the chemical functional groups of the group A are chosen from anhydride, epoxide, chlorotriazine, aldehyde and thiosulphate functional groups.

8. The process according to claim 1, wherein the chemical functional groups of the group B are chosen from hydroxyl, primary and secondary amine, thiol and carboxylic acid functional groups.

9. The process according to claim 1, wherein the at least one polymer PA and the at least one polymer PB each comprise at least two identical chemical functional groups chosen from the group A and the group B.

10. The process according to claim 1, wherein the at least one polymer PA and the at least one polymer PB are chosen from the following combinations:
dendrimers comprising end groups chosen from OH, NH₂, SH and COOH of at least one generation in combination with at least one polymer chosen from polymers comprising functional groups chosen from anhydride units and polymers comprising at least one group chosen from epoxide and aldehyde functional groups,
synthetic polymers comprising hydroxyl functional groups, in combination with at least one polymer chosen from polymers comprising functional groups chosen from anhydride units and polymers comprising epoxide functional groups,
polyethyleneimines in combination with at least one polymer chosen from polymers comprising functional groups chosen from anhydride units and polymers comprising at least one group chosen from epoxide and aldehyde functional groups,
polyethyleneimine thiols, obtained by reacting polyethyleneimines with γ-butyrolactone, in combination with at least one polymer chosen from polymers comprising functional groups chosen from anhydride units and polymers comprising at least one group chosen from epoxide and aldehyde functional groups,
amino acids comprising functional groups chosen from free OH, NH₂, SH and COOH groups, in combination with at least one polymer chosen from polymers comprising functional groups chosen from anhydride units and polymers comprising at least one group chosen from epoxide and aldehyde functional groups,
polysaccharides chosen from natural and modified polysaccharides comprising functional groups chosen from OH, NH₂, SH and COOH in combination with at least one polymer chosen from polymers comprising functional groups chosen from anhydride units and polymers comprising at least one group chosen from epoxide and aldehyde functional groups,
polysaccharides chosen from natural and modified polysaccharides comprising aldehyde functional groups in combination with polymers chosen from synthetic and natural polymers comprising functional groups chosen from OH, NH₂, SH and COOH,
polysaccharides chosen from natural and modified polysaccharides comprising epoxy functional groups in combination with polymers chosen from synthetic and natural polymers comprising functional groups chosen from OH, NH₂, SH and COOH, and
polymers chosen from natural and synthetic polymers comprising carboxylic acid functional groups in combination with polymers chosen from synthetic and natural polymers comprising functional groups chosen from OH, NH₂ and SH, in the presence of at least one entity chosen from carbodiimides, acids, bases and enzymes.

11. The process according to claim 10, wherein the anhydride functional group is maleic anhydride.

12. The process according to claim 10, wherein the synthetic polymers comprising hydroxyl functional groups are chosen from polyvinyl alcohols.

13. The process according to claim 10, wherein the amino acids comprising functional groups chosen from free OH, NH₂, SH and COOH groups are polylysines.

14. The process according to claim 10, wherein the enzymes are chosen from esterase, lipase and protease.

15. The process according to claim 1, wherein the at least one polymer PA comprising chemical functional groups of the group A is chosen from:
methyl vinyl ether/maleic anhydride copolymer,
polyglycidyl methacrylate,
glycidyl polydimethylsiloxane,
epoxy polyamidoamine,
epoxydextran, and
polysaccharides and polyaldehydes obtained by oxidizing polysaccharides with NalO₄.

16. The process according to claim 1, wherein the at least one polymer PB comprising chemical functional groups of the group B is chosen from:
PAMAM dendrimer,
dendrimer containing hydroxyl functions,
PEI (polyethyleneimine),
PEI-thiol,
polylysine,
HP cellulose,
aminodextran,
aminocellulose,
PVA,
amino PVA,
chitosan,
CM and HP dextran, and
CM and HP chitosan.

17. The process according to claim 1, wherein the at least one polymer PA and the at least one polymer PB are present at a concentration ranging from 0.05% to 50% by weight, relative to the total weight of the composition.

18. The process according to claim 17, wherein the at least one polymer PA and the at least one polymer PB are present at a concentration ranging from 0.1% to 20% by weight, relative to the total weight of the composition.

19. The process according to claim 18, wherein the at least one polymer PA and the at least one polymer PB are present at a concentration ranging from 0.25% to 10% by weight, relative to the total weight of the composition.

20. The process according to claim 1, further comprising at least one cosmetic additive chosen from fixing polymers; thickeners; anionic, nonionic, cationic and amphoteric surfactants; fragrances; preserving agents; sunscreens; proteins; vitamins; provitamins; anionic, nonionic, cationic and amphoteric non-fixing polymers; mineral, plant and synthetic oils; ceramides; pseudoceramides; volatile and non-volatile, linearand cyclic, modified and unmodified silicones; pH regulators; oxidizing agents; reducing agents; inhibitors; and catalysts.

21. The process according to claim 1, wherein the cosmetically acceptable medium is chosen from water, at least one cosmetically acceptable solvent, and mixtures thereof.

22. The process according to claim 21, wherein the at least one cosmetically acceptable solvent is chosen from alcohols, esters, ketones, and volatile cyclic silicones.

23. (canceled)

24. A method of forming a coat on hair comprising applying to the hair, separately or sequentially over time, at least two compositions, wherein one of the at least two compositions comprises at least one polymer PA, and the other of the at least two compositions comprises at least one polymer PB, respectively, wherein the at least one polymer PA and the at least one polymer PB are polymers comprising complementary chemical functional groups,
wherein the chemical functional groups of the group A are chosen from the following functional groups:
epoxide,
aziridine,
vinyl and activated vinyl,
anhydride, acid chloride and carboxylic acid esters,
aldehydes,
acetals, hemiacetals,
aminals, hemiaminals,
ketones, α-hydroxy ketones, α-halo ketones,
lactones, thiolactones,
isocyanate,
thiocyanate,
imines,
imides,
N-hydroxysuccinimide esters,
imidates,
thiosulphate,
oxazine and oxazoline,
oxazinium and oxazolinium,
C1 to C30 alkyl halides, C6 to C30 aryl and aralkyl halides of formula RX, wherein X is chosen from I, Br and Cl,
halides of unsaturated carbon-based rings and heterocycles,
sulphonyl halide chosen from RSO₂Cl and RSO₂F, and wherein R is chosen from C1 to C30 alkyls; and
wherein the chemical functional groups of the group B are chosen from functional groups of XHₙ, wherein X is chosen from O, N, S, and COO, and n=1 or 2.

25. A method according to claim 24, wherein the at least two compositions are packaged in a kit, wherein the kit comprises at least two compartments, the first compartment comprising, in a cosmetically acceptable medium, a composition comprising at least one polymer PA, and the second compartment comprising, in a cosmetically acceptable medium, at least one polymer PB, wherein the at least one polymer PA and the at least one polymer PB comprise complementary chemical functional groups, and the cosmetically acceptable mediums are identical or different.
